(19) United States
(12) Patent Application Publication
(10) Pub. No.: US 2020/0253584 A1
(43) Pub. Date: Aug. 13, 2020

(54) ULTRASONIC TRANSDUCER, DIAGNOSTIC ULTRASONIC PROBE, SURGICAL INSTRUMENT, SHEET-TYPE ULTRASONIC PROBE, AND ELECTRONIC APPARATUS

(71) Applicant: Sony Corporation, Tokyo (JP)

(72) Inventor: Rui MORIMOTO, Kanagawa (JP)

(21) Appl. No.: 16/637,581

(22) PCT Filed: Jun. 8, 2018
(86) PCT No.: PCT/JP2018/022074

§ 371 Date: Feb. 7, 2020

(30) Foreign Application Priority Data
Aug. 9, 2017 (JP) 2017-153865

(51) Int. Cl. A61B 8/00; B06B 1/06; A61B 10/06; A61B 8/12; G01N 29/24; G01S 15/89; G01S 7/524

(52) U.S. Cl. CPC A61B 8/4494; B06B 1/067; A61B 10/06; B06B 2201/76; G01N 29/2437; G01S 15/8906; G01S 7/524; A61B 8/12

(57) ABSTRACT

[Object] To provide an ultrasonic transducer, a diagnostic ultrasonic probe, a surgical instrument, a sheet-type ultrasonic probe, and an electronic apparatus by which both of favorable reflection characteristics and suppression of reverberation at low cost can be achieved.

[Solving Means] An ultrasonic transducer for ultrasonic imaging according to the present technology includes a piezoelectric layer, an acoustic attenuation layer, and an acoustic reflection layer. The piezoelectric layer is formed of a piezoelectric material and generates ultrasonic waves. The acoustic attenuation layer is formed of an acoustic attenuation material having an acoustic impedance lower than that of the piezoelectric material. The acoustic reflection layer is arranged on a side of the acoustic attenuation layer and which is formed of an acoustic reflection material having an acoustic impedance higher than that of the acoustic attenuation material, the side being opposite to the piezoelectric layer. The acoustic attenuation layer has a thickness which is integer multiple of ½ of a wavelength of an ultrasonic wave generated in the piezoelectric layer, the wavelength being inside the acoustic attenuation layer.

### TECHNICAL FIELD

The present technology relates to an ultrasonic transducer, a diagnostic ultrasonic probe, a surgical instrument, a sheet-type ultrasonic probe, and an electronic apparatus for the use in ultrasonic imaging.

### BACKGROUND ART

In ultrasonic imaging, ultrasonic waves are radiated from an ultrasonic probe including an ultrasonic transducer to an object to be imaged and reflection waves are searched for by using the ultrasonic probe. In this manner, an ultrasonic image of the object to be imaged is generated. Ultrasonic imaging enables a biological tissue to be seen through and is suitable for grasping a path of a blood vessel or position and shape of a tumor, finding nerves associated with a blood vessel, etc.

The ultrasonic transducer includes a piezoelectric layer formed of a piezoelectric material. The piezoelectric layer generates ultrasonic vibration in response to a driving signal. Further, when reflected waves generated in the object to be imaged arrive at the piezoelectric layer, a detection signal is generated and an ultrasonic image is generated on the basis of this detection signal.

In ultrasonic imaging, a technology (e.g., see Patent Literature 1) called dematching or heavy backing is used for improving a transmission sound pressure in recent years. In this technology, a member formed of a material (e.g., metal tungsten: to 105 MRayls) having a much higher acoustic impedance than that of a material (e.g., PZT: to 30 MRayls) of a piezoelectric element is arranged on a side opposite to an ultrasonic-wave transmission direction with respect to an ultrasonic transducer array.

Accordingly, sound waves are efficiently sent in the transmission direction by making an acoustic reflection at an interface larger. Even at the same application voltage, the sound pressure can be increased and the dynamic range can be enhanced. Further, sound waves that should be absorbed by backing (a member for absorbing unnecessary sound waves) decrease, and the heating value of heat that should be discharged also decreases. Therefore, the reliability in long-time use can also be enhanced.

Further, a technology called acoustic mirror is used in a high-frequency piezoelectric device. In this technology, thin films having different acoustic impedances are stacked as one layer to several tens of layers. A resonance structure is thus formed. With this structure, elastic waves are substantially completely reflected (e.g., see Patent Literature 2).

Elastic waves at several GHz are mainly used in such a high-frequency piezoelectric device. It can be fabricated by continuously depositing thin films each having a thickness of about several μm by physical vapor deposition (PVD) or chemical vapor deposition (CVD). Therefore, it is excellent in mass productivity and has been widely used.

### CITATION LIST

### Patent Literature

Patent Literature 1: Japanese Patent Application Laid-open No. 2010-148768
Patent Literature 2: Japanese Patent Application Laid-open No. 2004-159339

### DISCLOSURE OF INVENTION

### Technical Problem

However, tungsten used in the above-mentioned dematching technology is expensive (e.g., it costs ten times or more than stainless steel) and it is also difficult to work it. Therefore, the problem is an increase in cost. Further, the structure described in Patent Literature 1 is not a structure suitable for reducing the size and height of the ultrasonic transducer.

On the other hand, based on the theory of acoustic reflection, a large acoustic reflection is expected also in a material having a low acoustic impedance with respect to the piezoelectric material (e.g., PZT), for example, an inexpensive material like polyurethane (to 1.5 MRayls). At the same time, polyurethane has an acoustic absorption of 1 dB/mm/MHz or more and can be used also as an acoustic absorption material. Therefore, the number of components can be reduced in comparison with the structure described in Patent Literature 1. However, in general, a material having a low acoustic impedance is very soft. It is difficult to perform working such as dicing. It is also difficult to retain the structure.

Further, the structure described in Patent Literature 2 is assumed to use elastic waves at several GHz as described above. This structure is not often used in a frequency band of approximately 1 to 20 MHz to be used in ultrasonic imaging.

A reason is as follows. Specifically, pulse waves are generally used in ultrasonic imaging unlike the high-frequency piezoelectric device. With the resonance structure, favorable reflection characteristics cannot be obtained. An unintended reverberation is generated. An adverse effect is imposed on a dead zone (region in which imaging cannot be performed), a space resolution, a dynamic range, and an artifact (mankind's impact).

In view of the above-mentioned circumstances, it is an object of the present technology to provide an ultrasonic transducer, a diagnostic ultrasonic probe, a surgical instrument, a sheet-type ultrasonic probe, and an electronic apparatus by which both of favorable reflection characteristics and suppression of reverberation at low cost can be achieved.

### Solution to Problem

In order to accomplish the above-mentioned object, an ultrasonic transducer for ultrasonic imaging according to an embodiment of the present technology includes a piezoelectric layer, an acoustic attenuation layer, and an acoustic reflection layer.

The piezoelectric layer is formed of a piezoelectric material and generates ultrasonic waves.

The acoustic attenuation layer is formed of an acoustic attenuation material having an acoustic impedance lower than that of the piezoelectric material.

The acoustic reflection layer is arranged on a side of the acoustic attenuation layer and which is formed of an acoustic reflection material having an acoustic impedance higher than that of the acoustic attenuation material, the side being opposite to the piezoelectric layer.

The acoustic attenuation layer has a thickness which is integer multiple of ½ of a wavelength of an ultrasonic wave generated in the piezoelectric layer, the wavelength being inside the acoustic attenuation layer.

With this configuration, an acoustic impedance difference at an interface between the acoustic attenuation layer and the acoustic reflection layer. Therefore, a large acoustic reflection is generated at this interface and transmission ultrasonic waves are enhanced. Further, ultrasonic waves are confined in the acoustic attenuation layer. Therefore, acoustic enhancement on the lower frequency side is generated and the frequency band of ultrasonic waves is enhanced. In addition, the acoustic absorption efficiency is enhanced. Therefore, the acoustic attenuation layer can be thinned and the height of the ultrasonic transducer can be reduced.

The acoustic attenuation material may have an attenuation constant of 0.55 dB/mm/MHz or more.

In general, ultrasonic waves at 2 to 40 MHz are often used in ultrasonic imaging. By setting the acoustic attenuation material to have an attenuation constant of 0.55 dB/mm/MHz or more, a dead zone with ultrasonic waves at 2 to 40 MHz can be set to be equal to or smaller than a defined value.

The acoustic attenuation material may be a composite material having a resin material or a resin as a main material and including at least any of an organic compound, an inorganic compound, or a metal material.

The acoustic reflection material may be metal, an inorganic compound, or a composite material including metal and an inorganic compound.

A plurality of structures in which the acoustic attenuation layer and the acoustic reflection layer are stacked may be stacked.

With this configuration, multiple interfaces are formed between the acoustic attenuation layers and the acoustic reflection layers. Ultrasonic waves can be thus efficiently confined.

The acoustic reflection layer may be divided at a plurality of positions.

With this configuration, the ultrasonic transducer can be provided with plasticity.

In order to accomplish the above-mentioned object, a diagnostic ultrasonic probe according to an embodiment of the present technology includes an ultrasonic transducer for ultrasonic imaging. The ultrasonic transducer includes a piezoelectric layer, an acoustic attenuation layer, and an acoustic reflection layer.

The piezoelectric layer is formed of a piezoelectric material and generates ultrasonic waves.

The acoustic attenuation layer is formed of an acoustic attenuation material having an acoustic impedance lower than that of the piezoelectric material.

The acoustic reflection layer is arranged on a side of the acoustic attenuation layer and is formed of an acoustic reflection material having an acoustic impedance higher than that of the acoustic attenuation material, the side being opposite to the piezoelectric layer.

The acoustic attenuation layer has a thickness which is integer multiple of ½ of a wavelength of an ultrasonic wave generated in the piezoelectric layer, the wavelength being inside the acoustic attenuation layer.

In order to accomplish the above-mentioned object, a surgical instrument according to an embodiment of the present technology includes an ultrasonic transducer for ultrasonic imaging. The ultrasonic transducer includes a piezoelectric layer, an acoustic attenuation layer, and an acoustic reflection layer.

The piezoelectric layer is formed of a piezoelectric material and generates ultrasonic waves.

The acoustic attenuation layer is formed of an acoustic attenuation material having an acoustic impedance lower than that of the piezoelectric material.

The acoustic reflection layer is arranged on a side of the acoustic attenuation layer and is formed of an acoustic reflection material having an acoustic impedance higher than that of the acoustic attenuation material, the side being opposite to the piezoelectric layer.

The acoustic attenuation layer has a thickness which is integer multiple of ½ of a wavelength of an ultrasonic wave generated in the piezoelectric layer, the wavelength being inside the acoustic attenuation layer.

In order to accomplish the above-mentioned object, a sheet-type ultrasonic probe according to an embodiment of the present technology includes an ultrasonic transducer for ultrasonic imaging. The ultrasonic transducer includes a piezoelectric layer, an acoustic attenuation layer, and an acoustic reflection layer.

The piezoelectric layer is formed of a piezoelectric material and generates ultrasonic waves.

The acoustic attenuation layer is formed of an acoustic attenuation material having an acoustic impedance lower than that of the piezoelectric material.

The acoustic reflection layer is arranged on a side of the acoustic attenuation layer, is formed of an acoustic reflection material having an acoustic impedance higher than that of the acoustic attenuation material, and is divided at a plurality of positions, the side being opposite to the piezoelectric layer.

The acoustic attenuation layer has a thickness which is integer multiple of ½ of a wavelength of an ultrasonic wave generated in the piezoelectric layer, the wavelength being inside the acoustic attenuation layer.

In order to accomplish the above-mentioned object, an electronic apparatus according to an embodiment of the present technology includes an ultrasonic transducer for ultrasonic imaging. The ultrasonic transducer includes a piezoelectric layer, an acoustic attenuation layer, and an acoustic reflection layer.

The piezoelectric layer is formed of a piezoelectric material and generates ultrasonic waves.

The acoustic attenuation layer is formed of an acoustic attenuation material having an acoustic impedance lower than that of the piezoelectric material.

The acoustic reflection layer is arranged on a side of the acoustic attenuation layer and is formed of an acoustic reflection material having an acoustic impedance higher than that of the acoustic attenuation material, the side being opposite to the piezoelectric layer.

The acoustic attenuation layer has a thickness which is integer multiple of ½ of a wavelength of an ultrasonic wave generated in the piezoelectric layer, the wavelength being inside the acoustic attenuation layer.

### Advantageous Effects of Invention

As described above, in accordance with the present technology, it is possible to provide an ultrasonic transducer, a diagnostic ultrasonic probe, a surgical instrument, a sheet-type ultrasonic probe, and an electronic apparatus by which both of favorable reflection characteristics and suppression of reverberation at low cost can be achieved. It should be noted that the effects described here are not necessarily limitative and any effect described in the present disclosure may be provided.

### BRIEF DESCRIPTION OF DRAWINGS

FIG. 1 A perspective view of an ultrasonic transducer according to the embodiment of the present technology.

FIG. 2 A perspective view of a partial configuration of the ultrasonic transducer.

FIG. 3 A cross-sectional view of the ultrasonic transducer.

FIG. 4 A graph showing transmission waveforms of ultrasonic transducers according to example and comparative example.

FIG. 5 A graph showing frequency characteristics of the ultrasonic transducer at the time of transmission.

FIG. 6 A graph showing frequency characteristics of the ultrasonic transducer at the time of reception.

FIG. 7 A table showing a reverberation time of an ultrasonic transducer according to an embodiment of the present technology at the time of transmission.

FIG. 8 An explanatory diagram of a wave motion of a back surface direction in a backing structure of the ultrasonic transducer.

FIG. 9 A graph showing reflected waves in a piezoelectric layer of the ultrasonic transducer.

FIG. 10 A table showing a dead zone at each frequency of the ultrasonic transducer.

FIG. 11 A graph showing a relationship between a thickness of an acoustic attenuation layer of an ultrasonic transducer according to an embodiment of the present technology and an acoustic attenuation constant.

FIG. 12 A graph showing a relationship between the thickness of the acoustic attenuation layer of the ultrasonic transducer and a transmission waveform.

FIG. 13 A graph showing a relationship between the thickness of the acoustic attenuation layer of the ultrasonic transducer and frequency characteristics.

FIG. 14 A schematic view showing a manufacturing method for the ultrasonic transducer according to the embodiment of the present technology.

FIG. 15 A schematic view showing a manufacturing method for the ultrasonic transducer.

FIG. 16 A cross-sectional view of the ultrasonic transducer including a plurality of acoustic attenuation layers and a plurality of acoustic reflection layers according to an embodiment of the present technology.

FIG. 17 A schematic view of an ultrasonic probe including an ultrasonic transducer according to an embodiment of the present technology.

FIG. 18 A schematic view of an ultrasonic catheter including the ultrasonic transducer.

FIG. 19 A schematic view of an ultrasonic endoscope including the ultrasonic transducer.

FIG. 20 A schematic view of an intraoperative ultrasound probe including the ultrasonic transducer.

FIG. 21 A schematic view of a surgical instrument including the ultrasonic transducer.

FIG. 22 A schematic view of robotic forceps including the ultrasonic transducer.

FIG. 23 A schematic view of a sheet-type ultrasonic probe including the ultrasonic transducer.

FIG. 24 A schematic view showing a mode of use of the sheet-type ultrasonic probe including the ultrasonic transducer.

FIG. 25 A schematic view showing a mode of use of the sheet-type ultrasonic probe including the ultrasonic transducer.

FIG. 26 A schematic view of a smartphone including the ultrasonic transducer.

FIG. 27 A schematic view of a small authentication terminal including the ultrasonic transducer.

FIG. 28 A schematic view of an ATM including the ultrasonic transducer.

FIG. 29 A schematic view of an entry and exit system including the ultrasonic transducer.

### MODE(S) FOR CARRYING OUT THE INVENTION

An ultrasonic transducer according to each of the present embodiments will be described.

[Configuration of Ultrasonic Transducer]

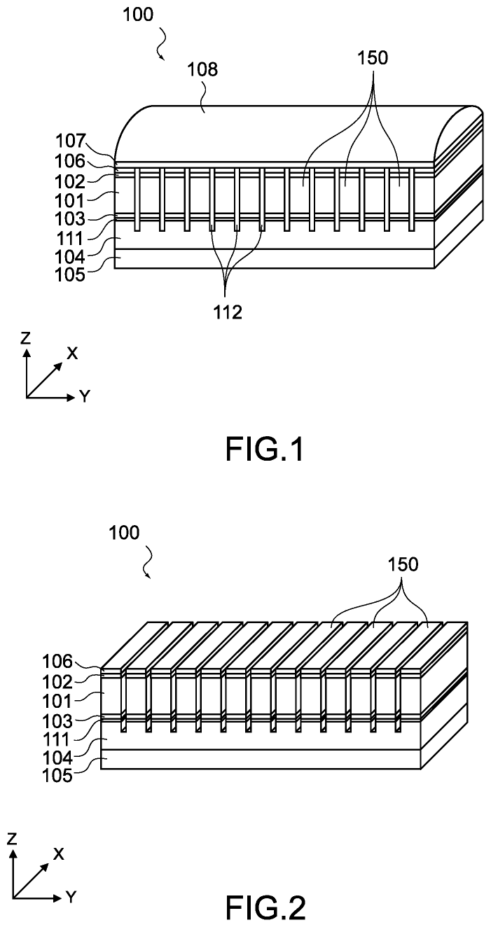

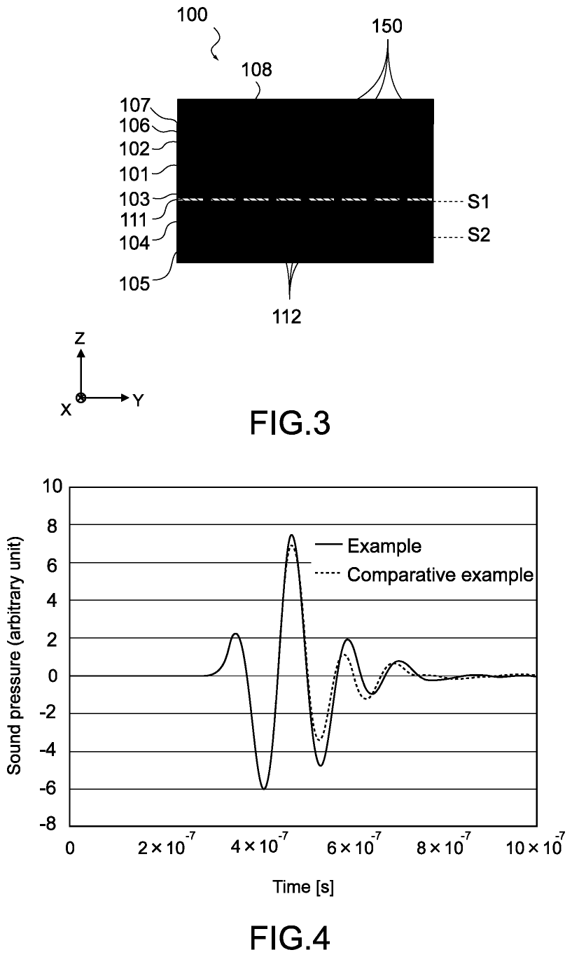

FIG. 1 is a perspective view of an ultrasonic transducer 100 according to this embodiment. FIG. 2 is a perspective view of a partial configuration of the ultrasonic transducer 100. FIG. 3 is a cross-sectional view of the ultrasonic transducer 100. In each of the drawings, three directions orthogonal to one another will be referred to as an X direction, a Y direction, and a Z direction.

As shown in FIGS. 1 to 3, the ultrasonic transducer 100 includes a piezoelectric layer 101, an upper electrode layer 102, a lower electrode layer 103, an acoustic attenuation layer 104, an acoustic reflection layer 105, a first acoustic matching layer 106, a second acoustic matching layer 107, and an acoustic lens 108.

As shown in FIGS. 2 and 3, the piezoelectric layer 101, the upper electrode layer 102, the first acoustic matching layer 106, the lower electrode layer 103, and the acoustic attenuation layer 104 are partially separated from each other. Each of them constitutes vibrator elements 150. That is, the ultrasonic transducer 100 is an array of the vibrator elements 150. A space between the respective vibrator elements 150 is filled with a kerf filler 112. Alternatively, the space between the respective vibrator elements 150 may be a gap.

The piezoelectric layer 101 is a piezoelectric material such as lead zirconate titanate (PZT: acoustic impedance to 30 MRayls). The piezoelectric layer 101 is provided between the lower electrode layer 103 and the upper electrode layer 102. When voltage is applied between the lower electrode layer 103 and the upper electrode layer 102, the piezoelectric layer 101 generates vibration due to the inverse piezoelectric effect and generates ultrasonic waves. Further, when the reflection waves from the object to be imaged enter the piezoelectric layer 101, polarization due to the piezoelectric effect is caused. Although the size of the piezoelectric layer 101 is not particularly limited, the size of the piezoelectric layer 101 can be 250 square μm, for example.

The upper electrode layer 102 is provided on the piezoelectric layer 101. The upper electrode layer 102 is formed of an electrically conductive material. The upper electrode layer 102 is metal deposited by plating or sputtering, for example. It should be noted that the upper electrode layer 102 may be separated for each of the vibrator elements 150 as shown in FIG. 3 or does not need to be separated. A flexible wiring board including a grounding wire to be connected to the upper electrode layer 102 is provided between the upper electrode layer 102 and the first acoustic matching layer 106. The flexible wiring board is provided on the back side in the figure.

The lower electrode layer 103 is provided on the acoustic attenuation layer 104. The lower electrode layer 103 is formed of an electrically conductive material. The lower electrode layer 103 is metal deposited by plating, sputtering, or the like, for example. A flexible wiring board 111 including a signal wire to be connected to the lower electrode layer 103 is provided between the lower electrode layer 103 and the acoustic attenuation layer 104.

The acoustic attenuation layer 104 is a layer that absorbs and attenuates ultrasonic waves radiated from the piezoelectric layer 101. Hereinafter, the material of the acoustic attenuation layer 104 will be referred to as an acoustic attenuation material. The acoustic attenuation material is a material having an acoustic impedance lower than that of the piezoelectric material that constitutes the piezoelectric layer 101.

A composite material having a resin material or a resin as a main material and including at least any of an organic compound, an inorganic compound, or a metal material can be used as the acoustic attenuation material. For example, polyurethane (acoustic impedance: 5 MRayls), an epoxy resin, a silicone resin, a nylon-based resin, or the like can be used.

The acoustic reflection layer 105 reflects ultrasonic waves emitted from the piezoelectric layer 101 and passing through the acoustic attenuation layer 104. Hereinafter, the material of the acoustic reflection layer 105 will be referred to as an acoustic reflection material. The acoustic reflection material has an acoustic impedance higher than that of the acoustic attenuation material. Further, the acoustic reflection material is more favorably one having an acoustic impedance having higher than that of the piezoelectric material that constitutes the piezoelectric layer 101.

Metal, an inorganic compound, or a composite material including metal and an inorganic compound can be used as the acoustic reflection material. Regarding metal, stainless steel (acoustic impedance: 47 MRayls), tungsten (101 MRayls), molybdenum (64 MRayls), copper (41 MRayls), gold (62 MRayls), nickel (50 MRayls), titanium (68 MRayls), a tin plate (37 MRayls), and/or the like can be used, for example. Regarding ceramics, TiC (42 MRayls), AIN (34 MRayls), SiN (36.2 MRayls), and/or the like can be used, for example.

The first acoustic matching layer 106 and the second acoustic matching layer 107 reduce a difference in acoustic impedance between the object to be imaged and the vibrator elements 150 and prevents ultrasonic waves from being reflected to the object to be imaged. The first acoustic matching layer 106 and the second acoustic matching layer 107 are formed of a synthetic resin or ceramic material. As shown in FIG. 3, the first acoustic matching layer 106 may be separated for each of the vibrator elements 150 and the second acoustic matching layer 107 does not need to be separated, though not limited thereto.

The acoustic lens 108 is held in contact with the object to be imaged and converges ultrasonic waves generated in the piezoelectric layer 101. The acoustic lens 108 is formed of a silicone rubber and/or the like, for example. The size and shape of the acoustic lens 108 are not particularly limited.

[Operation of Ultrasonic Transducer]

An operation of the ultrasonic transducer 100 will be described. The lower electrode layer 103 is supplied with a driving signal. Then, the potential difference between the upper electrode layer 102 and the lower electrode layer 103 causes vibration due to the inverse piezoelectric effect in the piezoelectric layer 101 and ultrasonic waves are generated. The generated ultrasonic waves are sent to the object to be imaged via the first acoustic matching layer 106, the second acoustic matching layer 107, and the acoustic lens 108. It should be noted that pulse waves are favorable as the driving signal.

The reflected waves generated in the object to be imaged are received by the piezoelectric layer 101 via the acoustic lens 108, the second acoustic matching layer 107, and the first acoustic matching layer 106. Polarization is caused due to the piezoelectric effect in the piezoelectric layer 101 and a current (hereinafter, a detection signal) flows through a signal wire. An ultrasonic image is generated by performing signal processing on this detection signal.

Here, ultrasonic waves generated in the piezoelectric layer 101 by supplying the driving signal travel to an acoustic lens 108 (hereinafter, forward) and travels opposite to the acoustic lens 108 (hereinafter, rearward). The ultrasonic waves travelling rearward are not sent to the object to be imaged as they are and do not contribute to ultrasonic imaging. However, those ultrasonic waves can be sent to the object to be imaged by being reflected forward.

In the ultrasonic transducer 100, the ultrasonic waves of the ultrasonic waves generated in the piezoelectric layer 101, which travel rearward, are reflected at an interface (hereinafter, interface S1) between the piezoelectric layer 101 and the acoustic attenuation layer 104 and at an interface (hereinafter, interface S2) between the acoustic attenuation layer 104 and the acoustic reflection layer 105 (hereinafter, interface S2) (see FIG. 3).

As described above, the acoustic attenuation layer 104 is formed of a material having an acoustic impedance lower than that of the piezoelectric layer 101 and the acoustic reflection layer 105 is formed of a material having an acoustic impedance higher than that of the acoustic attenuation layer 104. Therefore, an acoustic impedance difference between the acoustic attenuation layer 104 and the acoustic reflection layer 105 is large. Accordingly, a large acoustic reflection is generated at the interface S2 and ultrasonic waves reflected forward are enhanced.

Further, reflection of ultrasonic waves that travel rearward on the interface S1 and the interface S2 confines most of the ultrasonic waves in the acoustic attenuation layer 104. Accordingly, acoustic enhancement is caused on a lower frequency side and a frequency band of ultrasonic waves is widened.

As described above, with the ultrasonic transducer 100, ultrasonic waves sent to the object to be imaged can be enhanced and the frequency band can be widened by forwardly reflecting ultrasonic waves travelling rearward.

Further, the ultrasonic waves are confined in the acoustic attenuation layer 104, and thus the acoustic absorption efficiency at the acoustic attenuation layer 104 is enhanced. The thickness of the acoustic attenuation layer 104 can be thus reduced.

In addition, a material (polyurethane and/or the like) having an acoustic impedance smaller than that of the acoustic attenuation layer 104 generally has low rigidity, and thus it is difficult to maintain the ultrasonic transducer structure alone. However, the acoustic attenuation layer 104 is stacked on the acoustic reflection layer 105. The acoustic reflection layer 105 is formed of a material (stainless steel and/or the like) having a high acoustic impedance and the material having a high acoustic impedance generally has high rigidity, and thus the acoustic reflection layer 105 can maintain the structure of the ultrasonic transducer 100.

Further, the ultrasonic transducer 100 can be fabricated using an inexpensive material excellent in workability, such as polyurethane and stainless steel without the need for using a material having an ultra high acoustic impedance, such as tungsten. The cost can be thus reduced.

[Regarding Thickness of Acoustic Attenuation Layer]

The thickness of the acoustic attenuation layer 104 is favorably integer multiple of ½ of the wavelength of the ultrasonic waves generated in the piezoelectric layer 101, the wavelength being inside the acoustic attenuation layer 104. Accordingly, ultrasonic waves, which travel toward the interface S2 from the interface S1, are reflected on the interface S2, and arrive at the interface S1 again, are deviated by one wavelength or a plurality of wavelengths.

Therefore, the phase of ultrasonic waves that travel forward from the piezoelectric layer 101 and the phase of ultrasonic waves reflected on the interface S2 are identical to each other. When the ultrasonic waves overlap each other, the ultrasonic waves are enhanced. Therefore, the thickness of the acoustic attenuation layer 104 is favorably integer multiple of ½ of the wavelength of ultrasonic waves, the wavelength being inside the acoustic attenuation layer 104.

[Detection of Transducer Characteristics of Ultrasonic Transducer]

With respect to ultrasonic transducers according to an example and a comparative example, transducer characteristics are detected. The ultrasonic transducer 100 according to the example includes the acoustic attenuation layer 104 formed of an acoustic attenuation material (acoustic impedance: 5 MRayls) having a thickness of 0.2 mm and polyurethane as a main component and an acoustic reflection layer 105 which is a stainless steel plate (acoustic impedance: 47 MRayls) having a thickness of 0.1 mm.

A total thickness of the backing structure (the acoustic attenuation layer 104 and the acoustic reflection layer 105) is 0.3 mm and a total thickness of the ultrasonic transducer 100 including the backing structure to the acoustic lens 108 is 0.55 mm. This thickness achieves a reduction in height in comparison with the existing ultrasonic transducer.

Further, the ultrasonic transducer according to the comparative example has a structure excluding the acoustic reflection layer 105 from the ultrasonic transducer 100 according to the example.

FIG. 4 is transmission waveform results at 7 MHz of the ultrasonic transducers according to the example and the comparative example. In the example, the maximum sound pressure is enhanced by about 8% and the height of secondary waves is enhanced by about 15%. It implies that the ultrasonic transducer 100 according to the example has a large acoustic reflection.

FIG. 5 is a graph showing frequency characteristics of the ultrasonic transducers according to the example and the comparative example at the time of transmission at 7 MHz. As shown in the figure, in the example, the frequency band is widened in a low frequency wave direction in comparison with the comparative example. It results from enhancement of mainly secondary waves due to the provision of the acoustic reflection layer 105.

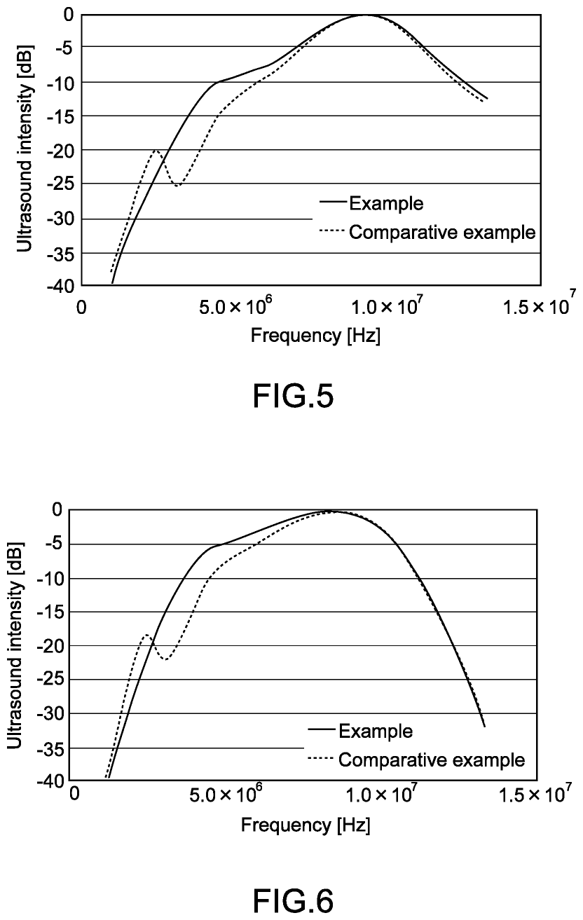

FIG. 6 is a graph showing frequency characteristics of reception sensitivity of the ultrasonic transducers according to the example and the comparative example. As shown in the figure, in the example, the frequency band is widened in the low frequency wave direction in comparison with the comparative example. It is because especially low frequency waves of reception waves are favorably confined in the backing structure.

On the other hand, in a case where strong acoustic confinement immediately below the ultrasonic transducer occurs, deterioration of the spatial resolution and the like due to reverberation are conceivable. However, such a problem is overcome by using an acoustic absorption material having high acoustic absorption characteristics such as polyurethane, for example.

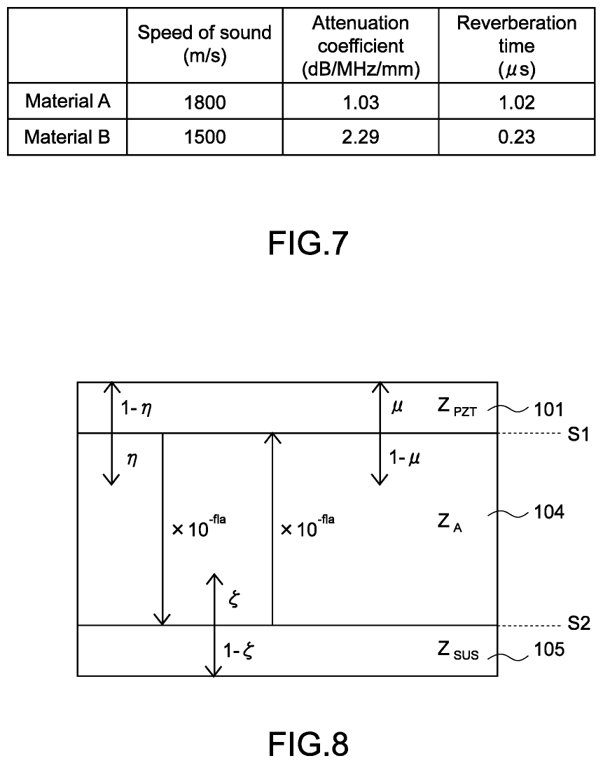

FIG. 7 is a table showing a reverberation time of the ultrasonic transducer 100 at the time of transmission at 7 MHz. As the material of the acoustic attenuation layer 104, a material A is a composite material of epoxy and tungsten and a material B is a material mainly containing polyurethane.

The reverberation time is a time difference in lowering of the output from the maximum of the transmission waveform to −20 dB. The thickness of the acoustic attenuation layer 104 is set to 0.2 mm and a stainless steel plate having a thickness of 0.1 mm is provided as the acoustic reflection layer 105.

Influence of reverberation is most prominent in a dead zone. The dead zone refers to a phenomenon where a portion closest to the ultrasonic transducer 100 cannot be imaged due to transmission waves. An allowable criteria thereof is 3.0 mm or less with transmission waves at about 7 MHz or more in accordance with the following reference document.

<Reference Document>

Mitchell M. Goodsitt et al. “Real-time B-mode ultrasound quality control test procedures Report of AAPM Ultrasound Task Group No.1”, Med. Phys., 25(8)(1998), p.1385-1406.

The material A and the material B both have acoustic attenuation characteristics of 1.0 dB/MHz/mm or more typically used as a backing material, and both are not a problem also in view of the dead zone.

Specifically, the material (acoustic attenuation material) of the acoustic attenuation layer 104 detects a favorable attenuation rate. FIG. 8 is an explanatory diagram of a wave motion of a back direction in the backing structure of the ultrasonic transducer 100.

In a case where the piezoelectric layer 101 is formed of PZT, transmittance η at an interface between the piezoelectric layer 101 and the acoustic attenuation layer 104 is expressed as Formula (1) below, assuming that an acoustic impedance of the PZT and the acoustic attenuation material is denoted by Z_{PZT} and Z_{A}, respectively.

η=2Z_{A}/(Z_{PZT}+Z_{A}) Formula (1)

Ultrasonic waves are attenuated in travelling inside the acoustic attenuation layer 104 and an attenuation rate ξ from the interface S1 to the interface S2 is expressed as Formula (2) below.

ξ=10^{-flα} Formula (2)

Here, l denotes a thickness of the acoustic attenuation layer 104, f denotes a frequency of ultrasonic waves emitted from the ultrasonic transducer, and α is an attenuation coefficient.

In addition, in a case where the acoustic reflection layer 105 is formed of stainless steel, the reflectance ζ at the interface S2 is expressed as Formula (3) below, assuming that the acoustic impedance of the acoustic reflection layer 105 is Z_{SUS}.

ζ=(Z_{SUS}−Z_{A})/(Z_{SUS}+Z_{A}) Formula (3)

In addition, a transmittanceμ and a reflectance 1−μ when ultrasonic waves pass through the interface S1 from the interface S2 are expressed as Formula (4) and Formula (5) below.

μ=2Z_{PZT}/(Z_{PZT}+Z_{A}) Formula (4)

1−μ=(Z_{A}−Z_{PZT})/(Z_{PZT}+Z_{A}) Formula (5)

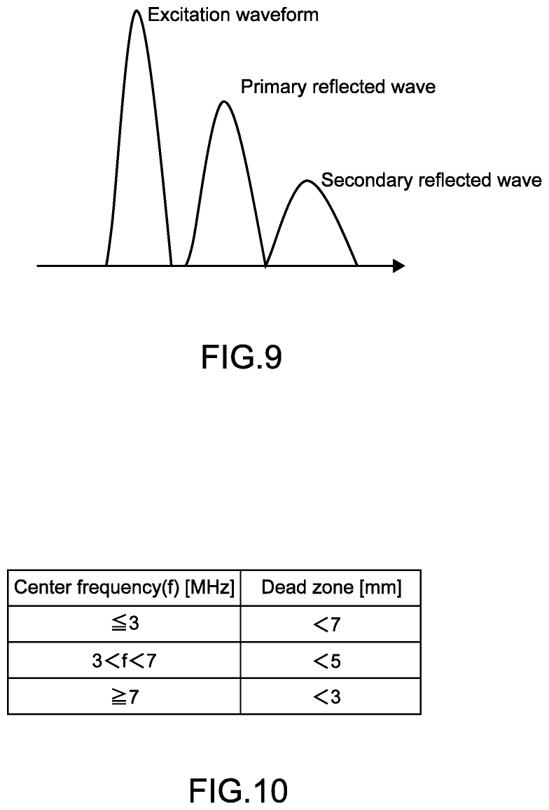

Reflected waves from the interface S2 experience multipath reflection inside the acoustic attenuation layer 104 and are attenuated in the piezoelectric layer 101. FIG. 9 is a graph showing reflected waves in the piezoelectric layer 101. An amplitude An of the nth reflection waves is expressed as Formula (6) below, assuming that an amplitude of excitation waveforms is denoted by A₀.

$${A}_{n} = {A}_{0} \times η * {ξ}^{2} * ζ * μ = {A}_{0} \times {\{ 2 {Z}_{A} / ( {Z}_{PZT} + {Z}_{A} ) * {10}^{- 2 fl α} * ( {Z}_{SUS} - {Z}_{A} ) / ( {Z}_{SUS} + {Z}_{A} ) \}}^{n} * {\{ ( {Z}_{A} - {Z}_{PZT} ) / ( {Z}_{PZT} + {Z}_{A} ) \}}^{n - 1} * 2 {Z}_{PZT} / ( {Z}_{PZT} + {Z}_{A} ) \qquad Formula ( 6 )$$

On the other hand, assuming that the thickness of the dead zone is denoted by d_{dead} and the speed of sound inside the object to be imaged (e.g., living body) is denoted by c_{B}, a time duration to corresponding thereto is expressed as Formula (7) below.

t_{d}=d_{dead}/c_{B} Formula (7)

In a case where the pulse width that produces a dead zone is defined by a time difference between a time at which a main body pulse is maximum and a time at which it lowers by −20 dB, it substantially equals that defined by the reverberation time in lowering by 20 dB. In a case where the amplitude lowers by −20 dB or more in Formula (6), Formula (8) below is satisfied.

Aₙ/A₀≤0.1 Formula (8)

That is, Formula (9) below is satisfied.

{2Z_{A}/(Z_{PZT}+Z_{A})*10^{-2flα}*(Z_{SUS}−Z_{A})/(Z_{SUS}+Z_{A})}ⁿ*{(Z_{A}−Z_{PZT})/(Z_{PZT}+Z_{A})}ⁿ⁻¹* 2Z_{PZT}/(Z_{PZT}+Z_{A})≤0.1 Formula (9)

In addition, a time tₙ at which the nth reflection waves are detected at the piezoelectric layer 101 is expressed as follows, assuming that the thickness of the acoustic attenuation layer 104 is denoted by l and the speed of sound at the acoustic attenuation layer 104 is denoted by cₐ.

tₙ=2nl/cₐ Formula (10)

The thickness of the dead zone d_{dead} has a constraint. Therefore, assuming that this constraint is denoted by dₜₕ, Formula (7) to Formula (11) as follows can be obtained.

tₜₕ=dₜₕ/c_{B} Formula (11)

Further, Formula (9) only needs to be established at the point of time tₜₕ. Therefore, Formula (12) below can be obtained on the basis of Formula (10) and Formula (11).

n=cₐdₜₕ/2c_{B}1 Formula (12)

By substituting Formula (12) into Formula (9) and modifying it into the formula of α, Formula (13) below can be obtained.

$$\begin{matrix} [ Expression 1 ] & \\ α \geq \frac{{c}_{B}}{{c}_{a} {d}_{t h} f} + \frac{1}{2 lf} log \frac{2 {Z}_{A} ( {Z}_{SUS} - {Z}_{A} ) ( {Z}_{A} - {Z}_{P Z T} )}{{( {Z}_{P Z T} + {Z}_{A} )}^{2} ( {Z}_{SUS} + {Z}_{A} )} + log \frac{2 {Z}_{P Z T}}{{Z}_{A} - {Z}_{P Z T}} & Formula ( 13 )\end{matrix}$$

Here, attempts are made regarding all case studies of ultrasonic imaging. In general, it is often performed in a range of 1 to 40 MHz in the ultrasonic imaging. The dead zone at each frequency is as shown in the table shown in FIG. 10 in accordance with the above-mentioned reference document.

Further, the speed of sound in a human soft tissue was set as 1450 to 1590 m/s, the speed of sound of each material used in the acoustic attenuation material was set as 800 to 3000 m/s, the acoustic impedance of each material used as the acoustic attenuation material was set as 1.5 to 10 MRayls, and the acoustic impedance of the acoustic reflection material was set to be a value higher than that of the acoustic attenuation material.

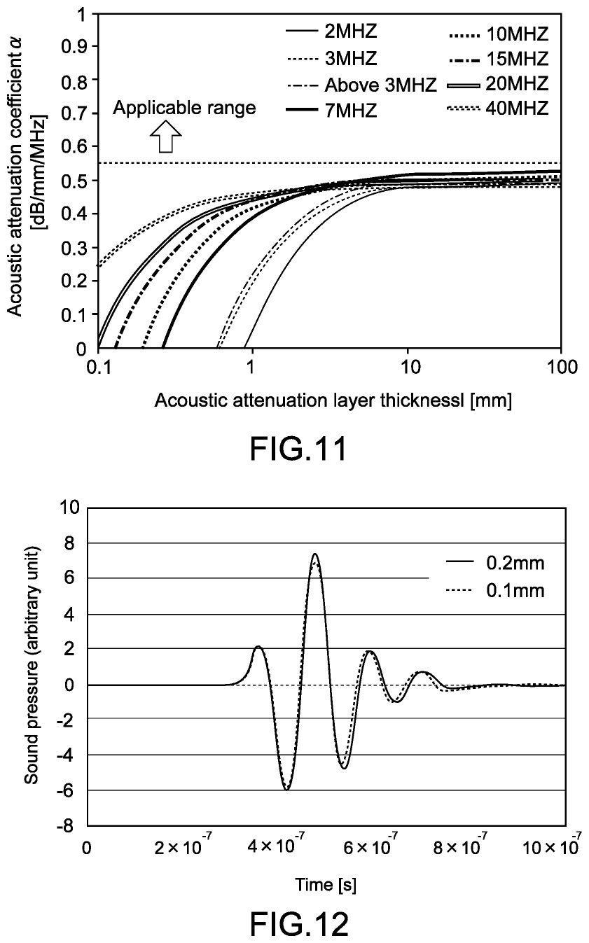

FIG. 11 is a graph showing a relationship between the thickness of the acoustic attenuation layer 104 and an acoustic attenuation constant. The horizontal axis indicates a thickness l of the acoustic attenuation layer 104 and the vertical axis indicates an attenuation constant α of the acoustic attenuation material. The trend line differs in a manner that depends on the frequency. In a frequency band of 2 MHz or more used in ultrasonic imaging, it can be said that the attenuation constant α of the acoustic attenuation material only needs to be 0.55 dB/MHz/mm or more.

At the same time, when the thickness of the acoustic attenuation layer 104 is designed such that the phase of transmission waves and the phase of the reflection waves from the interface S2 are aligned, the acoustic enhancement effect of transmission waves is enhanced. For example, assuming that the speed of sound inside the acoustic attenuation layer 104 is denoted by cₐ and a transmission frequency is denoted by f, the wavelength λ is expressed as Formula (14) below.

λ=cₐ/f Formula (14)

At this time, it is favorable that a sound channel length of ultrasonic waves which travel inside the acoustic attenuation layer 104 is integer multiple of the wavelength in order to increase the intensity of transmission waves. It is a first reflection wave of the interface S2 that mainly contributes to the intensity of transmission waves. Therefore, it is sufficient to take a sound channel length of the first reflection wave into consideration.

Assuming that the thickness of the acoustic attenuation layer 104 is denoted by l, a first sound channel length is expressed as 2l. Therefore, in a case where Formula (15) below is established, the phase of transmission waves and the phase of the reflection waves from the interface S2 can be aligned.

2l=mλ(m=1, 2, 3, . . . ) Formula (15)

On the other hand, in general, those having high acoustic attenuation characteristics are often used for the acoustic attenuation material. Therefore, maximum intensity contribution of transmission waves appears where m=1.

2l=λ Formula (16)

FIG. 12 is a graph showing this verification result. The figure shows sound-pressure comparison of a case where the thickness of the acoustic attenuation layer 104 is a half (0.2 mm) of the wavelength with a case where the thickness of the acoustic attenuation layer 104 is ¼ (0.1 mm) of the wavelength in the ultrasonic transducer 100. In the case where the wavelength is 1/4, the effect at the maximum sound pressure disappeared and the maximum sound pressure lowered at a level equal to or less than that in a case where the acoustic reflection layer 105 is not provided.

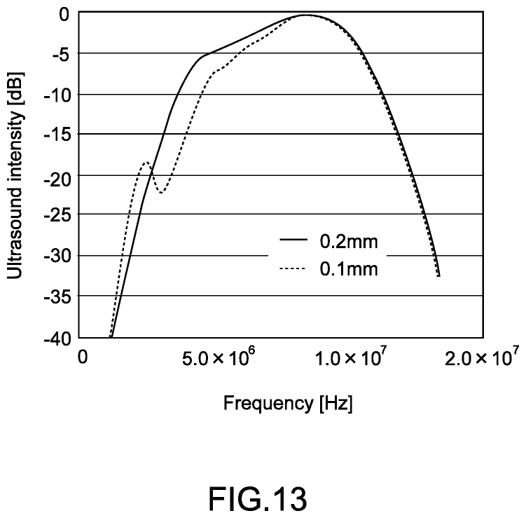

Further, FIG. 13 is a graph showing frequency characteristics of ultrasonic waves shown in FIG. 12. It can be seen that in the case where the wavelength is ¼ (0.1 mm), only an effect at a level equal to or less than that in a case where the acoustic reflection layer 105 is not provided can be obtained as in the case of the sound pressure.

As described above, in a case where the thickness of the acoustic attenuation layer 104 is integer multiple of ½ of the wavelength of ultrasonic waves inside the acoustic attenuation layer 104, enhancement of the transmission sound pressure of ultrasonic waves sent from the ultrasonic transducer 100 and widening of the frequency band are achieved.

[Manufacturing Method for Ultrasonic Transducer]

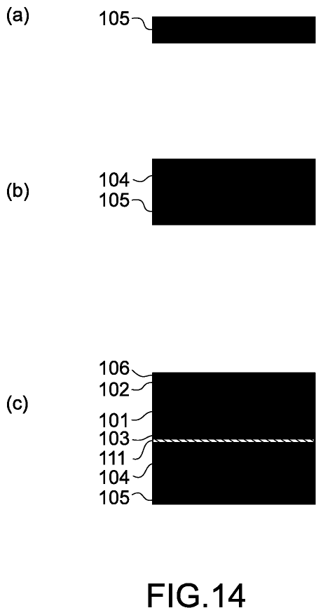

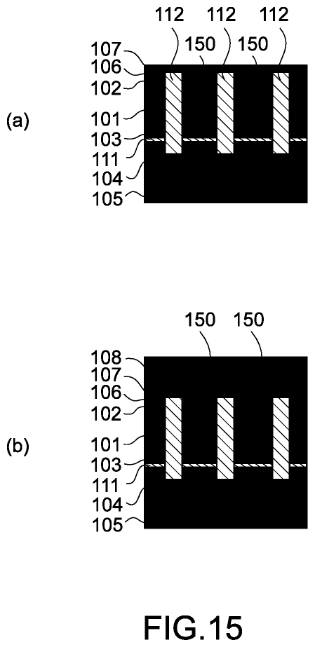

A manufacturing method for the ultrasonic transducer 100 will be described. FIGS. 14 and 15 are schematic views each showing a manufacturing method for the ultrasonic transducer 100.

First of all, as shown in (a) of FIG. 14, the acoustic reflection layer 105 is prepared. Next, as shown in (b) of FIG. 14, the acoustic attenuation layer 104 is arranged on the acoustic reflection layer 105.

Subsequently, as shown in (c) of FIG. 14, the piezoelectric layer 101 on which the lower electrode layer 103 and the upper electrode layer 102 are deposited is arranged above the acoustic attenuation layer 104. Further, the first acoustic matching layer 106 is arranged on the upper electrode layer 102.

Subsequently, as shown in (a) of FIG. 15, the first acoustic matching layer 106, the upper electrode layer 102, the piezoelectric layer 101, the lower electrode layer 103, and the acoustic attenuation layer 104 are partially diced and the individual vibrator elements 150 are formed. The gap between the vibrator elements 150 is filled with the kerf filler 112. In addition, the second acoustic matching layer 107 is arranged on the first acoustic matching layer 106.

Subsequently, as shown in (b) of FIG. 15, the acoustic lens 108 is arranged on the second acoustic matching layer 107. The ultrasonic transducer 100 can be fabricated in the above-mentioned manner. The fabrication method is not complicated even in comparison with the conventional one. At the same time, polyurethane can be used for the acoustic attenuation layer 104 and an inexpensive material such as stainless steel can be used for the acoustic reflection layer 105. Therefore, the ultrasonic transducer 100 can be fabricated at low cost.

[Regarding Number of Times of Stacking of Acoustic Attenuation Layer and Acoustic Reflection Layer]

In the above description, the ultrasonic transducer 100 has a structure in which the acoustic attenuation layer 104 and the acoustic reflection layer 105 are stacked. However, a plurality of structures in which the acoustic attenuation layer 104 and the acoustic reflection layer 105 are stacked may be stacked.

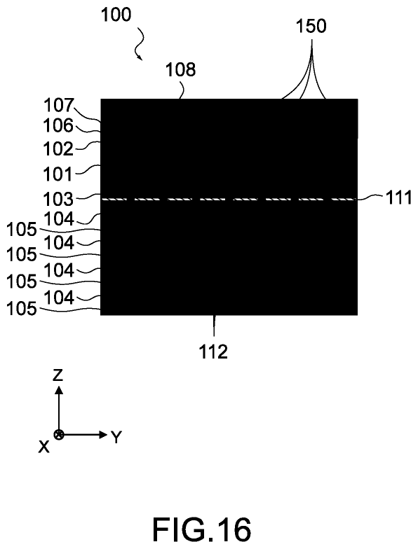

FIG. 16 is a cross-sectional view of the ultrasonic transducer 100 including the plurality of acoustic attenuation layers 104 and the acoustic reflection layer 105. As shown in the figure, the plurality of acoustic attenuation layers 104 and the plurality of acoustic reflection layers 105 are alternately stacked.

In a case where a plurality of structures in which the acoustic attenuation layer 104 and the acoustic reflection layer 105 are stacked is stacked, multiple interfaces are formed between the acoustic attenuation layers 104 and the acoustic reflection layers 105. Ultrasonic waves can be thus efficiently confined. Further, if the acoustic reflection layer 105 is sufficiently thin, it does not become a failure at the dicing step, and formation of a dematching and backing layer at low-price and with high performance is possible.

[Application Example 1: General Diagnostic Ultrasonic Probe]

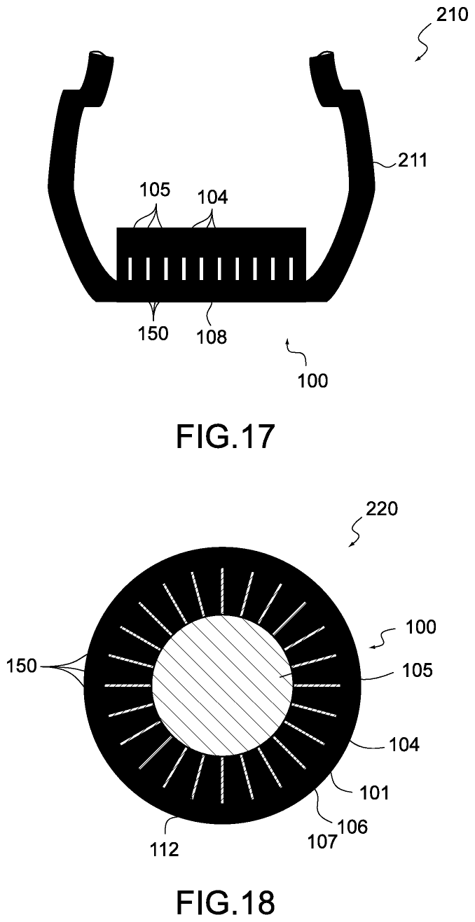

FIG. 17 is a schematic view of an ultrasonic probe 210 including the ultrasonic transducer 100. As shown in the figure, the ultrasonic probe 210 includes the ultrasonic transducer 100 housed in a probe casing 211.

As the general diagnostic ultrasonic probe, those that achieve acoustic intensity enhancement due to a dematching layer have been produced. With the present technology, the cost thereof can be reduced without largely changing the fabrication method. Further, heat dissipation characteristics can also be improved, and thus it is effective also in view of long-term reliability.

[Application Example 2: Ultrasonic Catheter]

FIG. 18 is a cross-sectional view of an ultrasonic catheter 220 including the ultrasonic transducer 100. As shown in the figure, the ultrasonic catheter 220 includes the ultrasonic transducer 100 formed in a linear shape having the acoustic reflection layer 105 as the center.

In the ultrasonic catheter, it is necessary to reduce the total diameter as much as possible in view of lowering of a risk of damage of an inner wall of a blood vessel and shortening of a hemostasis time. Conventionally, there is an example of coping with it by reducing the thickness of a sheath and/or the like (see the following reference document).

<Reference Document>https://www.terumo.co.jp/archive/p_ j/Presentation_130717_MTP_DDS_C&V_J_02.pdf

On the other hand, it is most effective to reduce the thickness of the backing structure occupying a large volume in the ultrasonic catheter for total diameter reduction. The ultrasonic catheter 220 shown in FIG. 18 can be a catheter for intravascular ultrasound (IVUS).

In the IVUS, ultrasonic waves at 20 to 40 MHz are normally used. A required thickness of the backing structure that absorbs it is 300 μm or more. Here, in accordance with the present technology, in a case where the acoustic attenuation layer 104 is made of a rubber such as polyurethane and the number of vibrations of ultrasonic waves is 20 MHz, the thickness only needs to be approximately 18 to 20 μm corresponding to a semi-wavelength. Further, in a case where the acoustic reflection layer 105 is formed of stainless steel, a diameter can be about 100 μm. Therefore, a total diameter can be reduced to 300 μm or less.

[Application Example 3: Ultrasonic Endoscope]

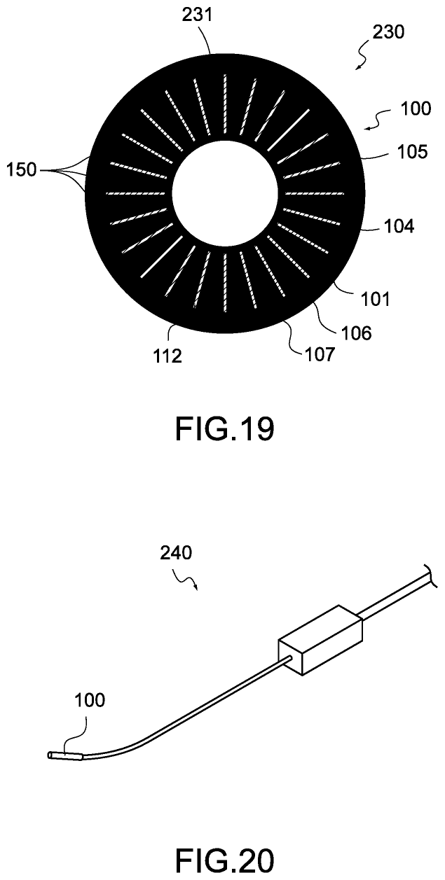

FIG. 19 is a cross-sectional view of an ultrasonic endoscope 230 including the ultrasonic transducer 100. As shown in the figure, the ultrasonic endoscope 230 includes a shaft 231 and the ultrasonic transducer 100 provided around the shaft 231.

Typically, the ultrasonic endoscope is classified into a small-diameter probe mainly for high-frequency image acquisition and convex-type and radial-type ultrasonic endoscope special-purpose machineries (see the following reference document).

<Reference Document>

SUGAWARA Toshiki, FUJITA Naotaka “Master US Today 2011 5. EUS 1) alimentary canal”, INNERVISION, Vol.26, 12 (2011), p. 46.

In general, an alimentary canal mucosa is observed using ultrasonic waves of 20 to 40 MHz in the small-diameter probe. While the burden on the patient in oral introduction owing to the reduced diameter, ultrasonic waves at 5 to 10 MHz are required for deep observation. However, in the conventional small-diameter probe, the diameter is about 3.2 mm and it is difficult to absorb ultrasonic waves in the above-mentioned range with the conventional backing structure.

On the other hand, sound waves at approximately 5 to 10 MHz can be absorbed with a thick backing structure in the convex-type ultrasonic endoscope and the radial-type ultrasonic endoscope. Therefore, it is also used by aspiration biopsy (EUS-FNA) with the ultrasonic image. However, the burden on the patient is relatively large because of an outer diameter of approximately 12 to 14 mm.

In a small-diameter probe-type ultrasonic endoscope 230 using the present technology shown in FIG. 16, the backing structure can be thinned and ultrasonic imaging at 5 to 10 MHz can be performed even with a small-diameter probe type. A stainless steel plate having a thickness of about 0.1 mm can be annular and it can be set as the acoustic reflection layer 105. A cut may be made in the stainless steel plate in order to ensure plasticity when the annular structure is fabricated.

Moreover, a forceps channel and a forceps opening through which a biopsy needle passes may be installed on a distal side from this ultrasonic transducer 100. With this structure, an image having a depth of permeation that enables the EUS-FNA to be performed can be obtained and applicable extension in the EUS-FNA and reduction in patient burden can be achieved.

[Application Example 4: Intraoperative Ultrasound Probe 1]

FIG. 20 is a cross-sectional view of an intraoperative ultrasound probe 240 including the ultrasonic transducer 100. As shown in the figure, the intraoperative ultrasound probe 240 includes the ultrasonic transducer 100.

In intraoperative ultrasonic imaging, a path of a blood vessel and an affected-part position inside a tissue is found using ultrasonic waves at approximately 5 to 10 MHz. In order to enable transmission of this ultrasonic wave to be performed, a thickness of 10 mm or more is generally set in the existing technology. In contrast, in accordance with the present technology, the backing structure can be made extremely thin even in a frequency range of 5 to 10 MHz.

Therefore, a further reduction in size/height can be achieved. For example, a reduction in size can be achieved to a size with which it can pass through a trocar having 5 mm Φ. Accordingly, minimally invasive surgery and alleviation of the burden on a patient can be achieved.

[Application Example 5: Surgical Instrument]

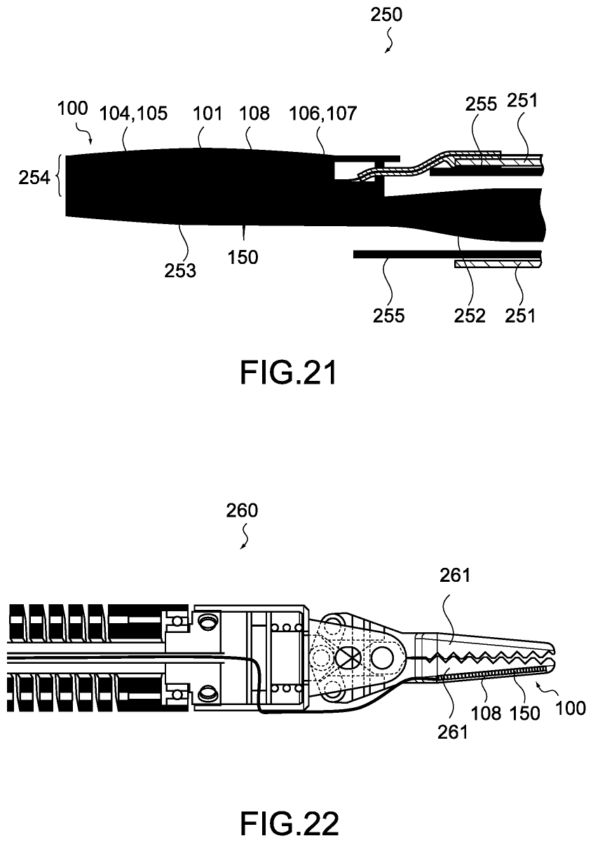

FIG. 21 is a cross-sectional view of a surgical instrument 250 including the ultrasonic transducer 100. As shown in the figure, the surgical instrument 250 includes a shaft 251, an ultrasonic-wave transfer bar 252, a blade 253, a movable jaw 254, a jaw-driving pipe 255, and the ultrasonic transducer 100.

The movable jaw 254 can be opened/closed with respect to the blade 253 by rotation of the jaw-driving pipe 255. A biological tissue can be pinched by the movable jaw 254 and the blade 253. The blade 253 applies ultrasonic waves to this pinched biological tissue and medical treatment such as cutting can be performed.

The ultrasonic transducer 100 is incorporated in the movable jaw 254. Ultrasonic imaging is configured to be performed by sending ultrasonic waves on a side opposite to the blade 253.

The movable jaw 254 is extremely thin. Therefore, it is extremely difficult to install the ultrasonic transducer. However, in accordance with the present technology, a further reduction in the ultrasonic transducer can be performed. Therefore, the ultrasonic transducer can be installed in the movable jaw 254.

In the surgical instrument 250 as shown in FIG. 21, the ultrasonic transducer is installed in the movable jaw 254 having a thickness of about 2 mm. In accordance with the present technology, it is possible to introduce the ultrasonic transducer also with such a thin component and to check a position of a blood-vessel or a portion to be incised in a deep area immediately before incision. Therefore, the safety and workability enhancement of surgery can be achieved.

[Application Example 6: Robotic Forceps]

FIG. 22 is a cross-sectional view of robotic forceps 260 including the ultrasonic transducer 100. As shown in the figure, the robotic forceps 260 includes a pinching portion 261 capable of pinching a biological tissue and the ultrasonic transducer 100 installed in the pinching portion 261.

For intraoperative ultrasonic imaging, various products have been sold. However, it is still difficult to check an incision position right before incision. Therefore, it is also conceivable to introduce the ultrasonic transducer 100 into the robotic forceps itself. On the other hand, a forceps using haptics as forceps for microsurgery in recent years is also conceivable. However, there is also a need for conveniently checking an inside of a surgery position, and the present technology can realize it.

[Application Example 7: Sheet-Type Ultrasonic Probe]

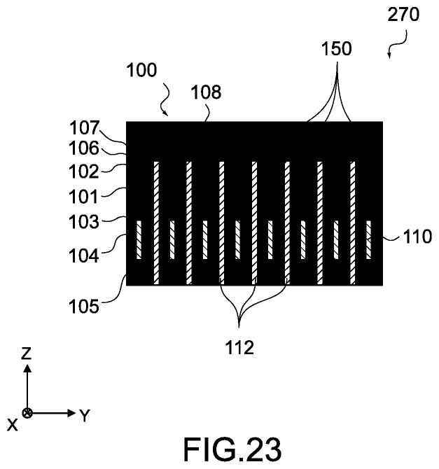

FIG. 23 is a cross-sectional view of a sheet-type ultrasonic probe 270 including the ultrasonic transducer 100. As shown in the figure, in the sheet-type ultrasonic probe 270, the acoustic attenuation layer 104 and the acoustic reflection layer 105 are also configured to be divided at a plurality of positions for each vibrator element 150. The lower electrode layer 103 and the acoustic reflection layer 105 are connected with wires 110 that penetrate the acoustic attenuation layer 104 and the acoustic reflection layer 105 functions as electrodes of the vibrator elements 150.

In particular, the ultrasonic transducer 100 can be provided with plasticity by dividing the acoustic reflection layer 105. The division of the acoustic reflection layer 105 can be performed by forming a kerf groove that arrives at the acoustic reflection layer 105 when the vibrator elements 150 are divided.

A sufficiently soft material mainly containing elastomer is favorable as the kerf filler 112. In addition, the plasticity of the ultrasonic transducer 100 is ensured and a sheet-shaped ultrasonic probe can be realized by using a flexible printed board for wiring.

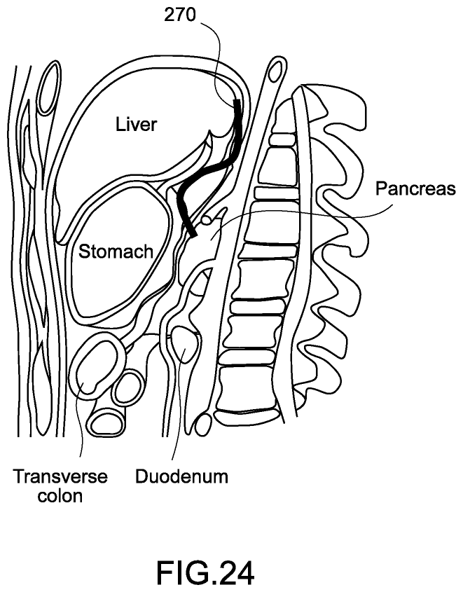

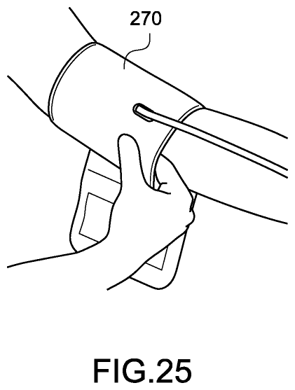

FIGS. 24 and 25 are schematic views each showing a mode of use of the sheet-type ultrasonic probe 270. As one of applications of the sheet-type ultrasonic probe 270, non-destructive examination having a tubular structure such as a water pipe is exemplified similar to the ultrasonic probe having the existing sheet shape.

Further, as a further application, it is also possible to enable surgery to be performed by arranging the sheet-type ultrasonic probe 270 below a liver during liver surgery as shown in FIG. 24, for example, while performing monitoring during surgery. In particular, in accordance with the present technology, relatively low frequency waves at 5 to 10 MHz can be sent even with a thin sheet-like structure. Therefore, it is possible to look out over a deep portion and in-situ observation of a biological tissue is useful in surgery. The workability of a surgery procedure and safety enhancement can be thus achieved.

Further, as shown in FIG. 25, the sheet-type ultrasonic probe 270 to be wound around the arm such as a cuff band can also be realized. There has been a problem in that the visibility of the ultrasonic image is changed and image interpretation and diagnosis easily vary in a manner that depends on procedures. However, the procedure dependency can be alleviated by the use of the present technology, and the stability of a diagnosis result and prevention of a wrong diagnosis can be achieved.

[Application Example 8: Biometrics Authentication]

The present technology can also be used in a biometric authentication technology. The biometric authentication technology performs identity verification on the basis of characteristics specific to an individual. Fingerprint authentication is generally used for a smartphone. In the fingerprint authentication, mainly an image sensor processes fingerprint image data.

However, in recent years, performance of a digital camera is enhanced, and an example in which a fingerprint is extracted from a snapshot or the like in which a fingertip has been captured is beginning to emerge. Such an example currently does not emerge in iris authentication. Along with technological progress of a digital camera, there is a possibility that this authentication technology is broken through.

<Reference Document>

OGANE takeo, ECHIZEN isao “BiometricJammer: Use of Pseudo Fingerprint to Prevent Fingerprint Extraction from Camera Images without Inconveniencing Users”, 2D1-3, computer security symposium 2016 Akita, Japan, 2016-10.

There is also a vein authentication technology using an infrared ray. With a near-infrared ray, extraction can be also performed by altering a commercially available camera. It is possible to check a vein having 1 to 2 mm under the skin in a contactless manner. For the purpose of avoiding it, there is an attempt to use a path of a blood vessel at a deeper position for biometrics authentication by introducing a complicated device structure and an algorithm. However, there are problems related to the cost and the like.

<Reference Document>

JP patent 597844

On the other hand, with ultrasonic waves at 10 to 15 MHz, it is extremely easy to obtain a depth of permeation exceeding 1 cm. In the present technology, an ultrasonic transducer having a small size and a small height can be formed. It is possible to realize a vein authentication system which can also be installed in a smartphone system and can easily achieve a depth of permeation of 1 cm or more.

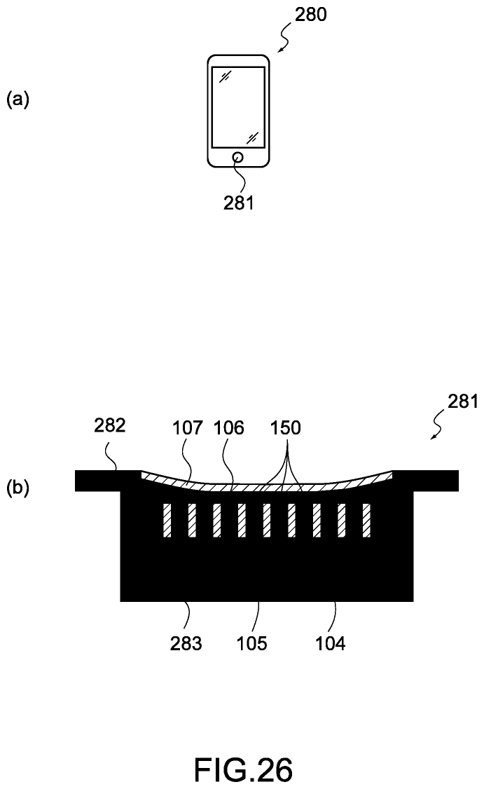

FIG. 26 is a schematic view of a smartphone 280 including the ultrasonic transducer 100. (A) of FIG. 26 is a plan view of the smartphone 280 and (b) of FIG. 26 is a cross-sectional view of a button 281 of the smartphone 280. As shown in (b) of FIG. 26, the button 281 includes a spring material 283 such as a rubber arranged in a supporting member 282 and the ultrasonic transducer 100 arranged on the spring material 283. The second acoustic matching layer 107 of the ultrasonic transducer 100 also serves as a button surface and is formed of a material easy to obtain matching between a living body and an acoustic impedance.

In accordance with the present technology, the thickness of the ultrasonic transducer is easily set to 0.5 mm or less. Therefore, the ultrasonic transducer can be installed in various mobile devices such as a smartphone for which excellent portability and design are required.

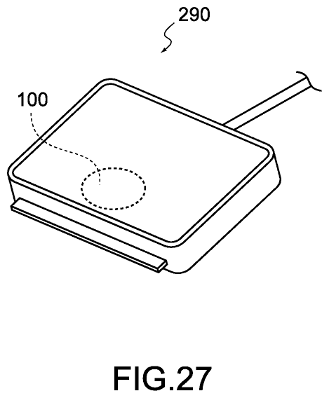

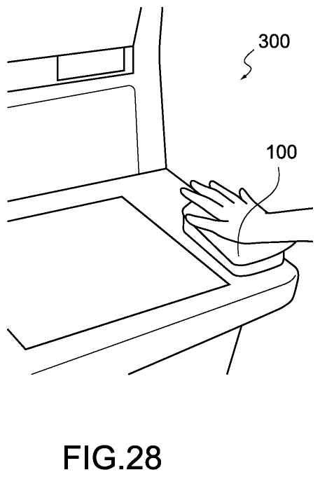

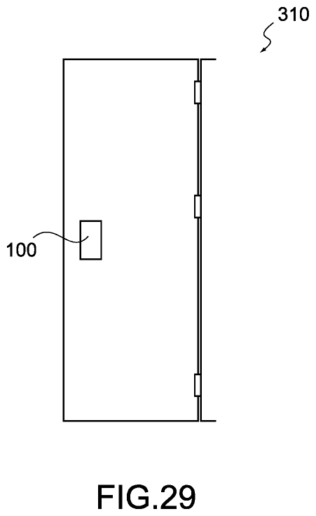

FIG. 27 is a schematic view of a small authentication terminal 290 for settlement by credit card or the like, which is placed at a retail store or the like. FIG. 28 is a schematic view of a bank automatic teller machine (ATM) 300. FIG. 29 is a schematic view of an entry and exit system 310 that manages the entry and exit of a house, an office, or the like.

The ultrasonic transducer 100 according to the present technology can also be installed in various biometric authentication devices as shown in those figure. In accordance with the present technology, it is easy to fabricate a sensor unit to be extremely thin and it is possible to improve the design of the device.

It should be noted that the present technology may also take the following configurations.

(1) An ultrasonic transducer for ultrasonic imaging, including:

a piezoelectric layer which is formed of a piezoelectric material and generates ultrasonic waves;

an acoustic attenuation layer formed of an acoustic attenuation material having an acoustic impedance lower than that of the piezoelectric material; and

an acoustic reflection layer which is arranged on a side of the acoustic attenuation layer and which is formed of an acoustic reflection material having an acoustic impedance higher than that of the acoustic attenuation material, the side being opposite to the piezoelectric layer, in which

the acoustic attenuation layer has a thickness which is integer multiple of ½ of a wavelength of an ultrasonic wave generated in the piezoelectric layer, the wavelength being inside the acoustic attenuation layer.

(2) The ultrasonic transducer according to (1), in which

the acoustic attenuation material has an attenuation constant of 0.55 dB/mm/MHz or more.

(3) The ultrasonic transducer according to (1) or (2), in which

the acoustic attenuation material is a composite material having a resin material or a resin as a main material and including at least any of an organic compound, an inorganic compound, or a metal material.

(4) The ultrasonic transducer according to any one of (1) to (3), in which

the acoustic reflection material is metal, an inorganic compound, or a composite material including metal and an inorganic compound.

(5) The ultrasonic transducer according to any one of (1) to (4), in which

a plurality of structures in which the acoustic attenuation layer and the acoustic reflection layer are stacked is stacked.

(6) The ultrasonic transducer according to any one of (1) to (5), in which

the acoustic reflection layer is divided at a plurality of positions.

(7) A diagnostic ultrasonic probe including an ultrasonic transducer for ultrasonic imaging, including:

a piezoelectric layer that is formed of a piezoelectric material and generates ultrasonic waves;

an acoustic attenuation layer that is formed of an acoustic attenuation material having an acoustic impedance lower than that of the piezoelectric material; and

an acoustic reflection layer that is arranged on a side of the acoustic attenuation layer and is formed of an acoustic reflection material having an acoustic impedance higher than that of the acoustic attenuation material, the side being opposite to the piezoelectric layer, in which

the acoustic attenuation layer has a thickness which is integer multiple of ½ of a wavelength of an ultrasonic wave generated in the piezoelectric layer, the wavelength being inside the acoustic attenuation layer.

(8) A surgical instrument including an ultrasonic transducer for ultrasonic imaging, including:

a piezoelectric layer that is formed of a piezoelectric material and generates ultrasonic waves;

an acoustic attenuation layer that is formed of an acoustic attenuation material having an acoustic impedance lower than that of the piezoelectric material; and

an acoustic reflection layer that is arranged on a side of the acoustic attenuation layer and is formed of an acoustic reflection material having an acoustic impedance higher than that of the acoustic attenuation material, the side being opposite to the piezoelectric layer, in which

the acoustic attenuation layer has a thickness which is integer multiple of ½ of a wavelength of an ultrasonic wave generated in the piezoelectric layer, the wavelength being inside the acoustic attenuation layer.

(9) A sheet-type ultrasonic probe including an ultrasonic transducer for ultrasonic imaging, including:

a piezoelectric layer that is formed of a piezoelectric material and generates ultrasonic waves;

an acoustic attenuation layer that is formed of an acoustic attenuation material having an acoustic impedance lower than that of the piezoelectric material; and

an acoustic reflection layer which is arranged on a side of the acoustic attenuation layer, is formed of an acoustic reflection material having an acoustic impedance higher than that of the acoustic attenuation material, and is divided at a plurality of positions, the side being opposite to the piezoelectric layer, in which

the acoustic attenuation layer has a thickness which is integer multiple of ½ of a wavelength of an ultrasonic wave generated in the piezoelectric layer, the wavelength being inside the acoustic attenuation layer.

(10) An electronic apparatus including an ultrasonic transducer for ultrasonic imaging, including:

a piezoelectric layer that is formed of a piezoelectric material and generates ultrasonic waves;

an acoustic attenuation layer that is formed of an acoustic attenuation material having an acoustic impedance lower than that of the piezoelectric material; and

an acoustic reflection layer that is arranged on a side of the acoustic attenuation layer and is formed of an acoustic reflection material having an acoustic impedance higher than that of the acoustic attenuation material, the side being opposite to the piezoelectric layer, in which

the acoustic attenuation layer has a thickness which is integer multiple of ½ of a wavelength of an ultrasonic wave generated in the piezoelectric layer, the wavelength being inside the acoustic attenuation layer.

### REFERENCE SIGNS LIST

100 ultrasonic transducer
101 piezoelectric layer
102 upper electrode layer
103 lower electrode layer
104 acoustic attenuation layer
105 acoustic reflection layer
106 first acoustic matching layer
107 second acoustic matching layer
108 acoustic lens
150 vibrator element
200 ultrasonic probe
210 ultrasonic probe
220 ultrasonic catheter
230 ultrasonic endoscope
240 intraoperative ultrasound probe
250 surgical instrument
260 robotic forceps
270 sheet-type ultrasonic probe
280 smartphone
290 small authentication terminal
300 bank ATM
310 entry and exit system

1. An ultrasonic transducer for ultrasonic imaging, comprising:
a piezoelectric layer which is formed of a piezoelectric material and generates ultrasonic waves;
an acoustic attenuation layer formed of an acoustic attenuation material having an acoustic impedance lower than that of the piezoelectric material; and
an acoustic reflection layer which is arranged on a side of the acoustic attenuation layer and which is formed of an acoustic reflection material having an acoustic impedance higher than that of the acoustic attenuation material, the side being opposite to the piezoelectric layer, wherein
the acoustic attenuation layer has a thickness which is integer multiple of ½ of a wavelength of an ultrasonic wave generated in the piezoelectric layer, the wavelength being inside the acoustic attenuation layer.

2. The ultrasonic transducer according to claim 1, wherein
the acoustic attenuation material has an attenuation constant of 0.55 dB/mm/MHz or more.

3. The ultrasonic transducer according to claim 2, wherein
the acoustic attenuation material is a composite material having a resin material or a resin as a main material and including at least any of an organic compound, an inorganic compound, or a metal material.

4. The ultrasonic transducer according to claim 1, wherein
the acoustic reflection material is metal, an inorganic compound, or a composite material including metal and an inorganic compound.

5. The ultrasonic transducer according to claim 1, wherein
a plurality of structures in which the acoustic attenuation layer and the acoustic reflection layer are stacked is stacked.

6. The ultrasonic transducer according to claim 1, wherein
the acoustic reflection layer is divided at a plurality of positions.

7. A diagnostic ultrasonic probe including an ultrasonic transducer for ultrasonic imaging, comprising:
a piezoelectric layer that is formed of a piezoelectric material and generates ultrasonic waves;
an acoustic attenuation layer that is formed of an acoustic attenuation material having an acoustic impedance lower than that of the piezoelectric material; and
an acoustic reflection layer that is arranged on a side of the acoustic attenuation layer and is formed of an acoustic reflection material having an acoustic impedance higher than that of the acoustic attenuation material, the side being opposite to the piezoelectric layer, wherein
the acoustic attenuation layer has a thickness which is integer multiple of ½ of a wavelength of an ultrasonic wave generated in the piezoelectric layer, the wavelength being inside the acoustic attenuation layer.

8. A surgical instrument including an ultrasonic transducer for ultrasonic imaging, comprising:
a piezoelectric layer that is formed of a piezoelectric material and generates ultrasonic waves;
an acoustic attenuation layer that is formed of an acoustic attenuation material having an acoustic impedance lower than that of the piezoelectric material; and
an acoustic reflection layer that is arranged on a side of the acoustic attenuation layer and is formed of an acoustic reflection material having an acoustic impedance higher than that of the acoustic attenuation material, the side being opposite to the piezoelectric layer, wherein
the acoustic attenuation layer has a thickness which is integer multiple of ½ of a wavelength of an ultrasonic wave generated in the piezoelectric layer, the wavelength being inside the acoustic attenuation layer.

9. A sheet-type ultrasonic probe including an ultrasonic transducer for ultrasonic imaging, comprising:
a piezoelectric layer that is formed of a piezoelectric material and generates ultrasonic waves;
an acoustic attenuation layer that is formed of an acoustic attenuation material having an acoustic impedance lower than that of the piezoelectric material; and
an acoustic reflection layer which is arranged on a side of the acoustic attenuation layer, is formed of an acoustic reflection material having an acoustic impedance higher than that of the acoustic attenuation material, and is divided at a plurality of positions, the side being opposite to the piezoelectric layer, wherein
the acoustic attenuation layer has a thickness which is integer multiple of ½ of a wavelength of an ultrasonic wave generated in the piezoelectric layer, the wavelength being inside the acoustic attenuation layer.

10. An electronic apparatus including an ultrasonic transducer for ultrasonic imaging, comprising:
a piezoelectric layer that is formed of a piezoelectric material and generates ultrasonic waves;
an acoustic attenuation layer that is formed of an acoustic attenuation material having an acoustic impedance lower than that of the piezoelectric material; and
an acoustic reflection layer that is arranged on a side of the acoustic attenuation layer and is formed of an acoustic reflection material having an acoustic impedance higher than that of the acoustic attenuation material, the side being opposite to the piezoelectric layer, wherein
the acoustic attenuation layer has a thickness which is integer multiple of ½ of a wavelength of an ultrasonic wave generated in the piezoelectric layer, the wavelength being inside the acoustic attenuation layer.
